(19) United States
(12) Patent Application Publication
(10) Pub. No.: US 2005/0164204 A1
(43) Pub. Date: Jul. 28, 2005

(54) SINGLE USE LYOPHILIZED RNASE REAGENTS, AND KITS AND METHODS FOR USING SAME

(76) Inventor: Thomas David Reed, Cincinnati, OH (US)

Correspondence Address: HASSE & NESBITT LLC, 7550 CENTRAL PARK BLVD., MASON, OH, 45040, US

(21) Appl. No.: 10/707,940

(22) Filed: Jan. 27, 2004

(51) Int. Cl. C12Q 1/68; C12M 1/34

(52) U.S. Cl. 435/6; 435/287.2

(57) ABSTRACT

A single use reagent article which comprises: (a) a holder; and a premeasured single use quantity of at least one lyophilized RNase associated with the holder. This reagent article can be used in a wide variety of biological kits and biological methods, such as molecular biological kits and methods for isolating DNA, where a known quantity of RNase(s) is needed having relatively consistent and stable activity. The reagent article is relatively easy to use and can minimize potential errors to due to user variability.

### BACKGROUND OF INVENTION

This invention relates to single use reagents comprising a premeasured quantity of at least one lyophilized RNase.

This invention further relates to kits and methods for using such reagents.

Ribonucleases (RNases) are proteolytic enzymes that selectively degrade RNA molecules, but generally does not recognize or degrade DNA molecules. Native RNases can be isolated from various cellular sources. Alternatively, the gene encoding the RNase can be isolated, amplified, and transferred to a host genome, propagated under conditions that promote production of the RNase, and then isolated using a series of standard protein purification steps. For example, the RNase isoform known as RNase T1 can be isolated from an over-expressing E. coli strain containing the cloned Aspergillus oryzae RNase T1-encoding gene. RNase T1 cleaves RNA after G residues and frequently is used for RNA mapping, as well as for some ribonuclease protection assay protocols.

In addition to RNase T1, there are other isoforms of RNase, including those known as RNase A, RNase I, RNase H, etc. RNase A cleaves RNA after the C and U residues. RNase I can degrade any RNA to a mixture of mono-, di-, and trinucleotides and has a marked preference for single-stranded RNA over double-stranded RNA, which allows it to work well in methods for analyzing RNA structure or abundance. While RNase I does not degrade DNA, it will bind to it. RNase H hydrolyzes RNA only in RNA:DNA hybrids, and will not degrade single-stranded DNA or RNA.

RNases are widely used by molecular biologists to remove contaminating RNA molecules from DNA isolates, and for controlled proteolytic analysis of RNA structure and function. Because each RNase has a different specificity for hydrolyzing RNA, they are thus useful for different applications. For example, RNase A is most commonly used to remove RNA that is contaminating plasmid preparations, as well as for digestion of unhybridized RNA in ribonuclease protection assays. However, digestion of RNA with RNase A alone can leave fragments of RNA which are large enough to be visible on agarose gels and to precipitate in ethanol. RNase H is commonly used to destroy an RNA template after first-strand cDNA synthesis. RNase mixtures can be used where it is desirable to degrade the RNA more thoroughly such as plasmid minipreps. For example, because RNase T1 cleaves RNA after the G residues, while RNase A cleaves RNA after C and U residues, mixtures of RNase T1 and RNase A can do better job of reducing RNA fragment size over the use of either RNase alone. Indeed, plasmid DNA isolation protocols frequently require the use of RNase mixtures to degrade contaminating RNA molecules as completely as possible.

Individual RNase isoforms and mixtures of RNases are commercially available from a variety of suppliers as lyophilized powders or in solutions. Typically, the lyophilized RNase is sold in bulk in small quantities, e.g. microgram to milligram amounts. The scientist then prepares a solution of a single RNase isoform or mixture thereof by resuspending a portion of the bulk quantity in an aqueous buffer at a concentration suitable for a particular application, and frequently divides the solution into aliquots for storage. These prepared RNase solutions are typically stored as refrigerated or frozen aliquots to protect the RNase enzymes from degradation.

Current preparations and delivery systems for providing RNase as a reagent for various molecular biological uses can create problems in terms of ease and convenience of use, ease of storage, avoidance of user variability that cause errors, as well as maintaining requisite RNase activity over time, i.e., shelf-life. The RNase is typically heated to 94° C. to destroy any contaminating DNase activity, and then cooled and stored at 20° C. or less. Some RNase solutions need to be stored as RNase/glycerol mixtures at 80° C. to preserve maximal activity. This requires the use of expensive cooling systems that may be unavailable to some users. The enzymatic activity (e.g., half-life) of RNase is also highly dependent upon proper handling, resuspension, and storage techniques. Activity is best preserved when the RNase is a lyophilized powder or is in a frozen solution. However, while readily soluble in wide variety of aqueous media, RNase is maximally active at a pH of from about 2 to about 4.5. Repeated freeze-thaw cycles can also decrease the activity of RNase.

There are also many commercially available kits for isolating DNA that include RNase or RNase mixtures as a component that is used to remove contaminating RNA from the DNA isolate. Typically, the RNA is supplied as a lyophilized bulk powder or as a bulk aqueous solution, and frequently includes 50% glycerol to inhibit enzyme activity during storage. For example, Ambion's RNase Cocktail™ comprises a mixture of two highly purified ribonucleases, RNase A and RNase T1 as a solution in 50% glycerol. The RNase powder or solution is typically added to another solution, such as a buffer solution that is used the initial step for isolation of plasmid DNA. This buffer can have a pH as high as 8.0 and is stored at 4° C. after addition of RNase in order to prolong shelf life. However, this is typically higher than the range for maximum stability of the RNase (i.e., pH of from about 2 to about 4.5). As a result, the shelf life is often compromised, and the RNase frequently does not efficiently degrade RNA when used in most plasmid isolation kits. Another significant limitation of many current DNA isolation methods involving bacteria is that the RNase does not readily come into contact with the RNA until the bacteria is subjected to an alkaline lysis step at a pH in excess of 8.0. Again, the relatively high pH of the alkaline lysis step is inhibitory to RNase activity, and thus decreases its effectiveness precisely when it comes into contact with the RNA.

Therefore, it would be desirable to develop a delivery system for RNase(s) at a later point in the DNA isolation process where inhibitory substances are not present, thereby greatly enhancing the ability of the RNase(s) to degrade contaminating RNA. It would also be desirable if such a delivery system could be easily incorporated into a variety of standard commercial kits for isolating DNA. It would be further desirable to develop a delivery system that would enhance the convenience, shelf-life and storage stability of RNase(s). In addition, it would be desirable to decrease errors in preparing RNase(s) reagents caused user variability so as to provide more predictable and consistent level of enzymatic activity.

### SUMMARY OF INVENTION

An embodiment of this invention relates to a single use reagent article for delivering a known quantity of at least one lyophilized RNase. This reagent article comprises: (a) a holder; and (b) a premeasured single use quantity of at least one lyophilized RNase associated with the holder.

Another embodiment of this invention relates to a biological kit. This kit comprises (a) at least one reagent article (as previously defined) as a component thereof; and (b) at least one other component selected from the group consisting of other reagents and reagent equipment.

Another embodiment of this invention relates to a method for removing contaminating RNA from a biological sample containing DNA. This method comprises the steps of a providing a biological sample containing DNA and contaminating RNA; b. providing a single use reagent article (as previously defined) wherein the lyophilized RNase(s) is capable of, and in a premeasured single use quantity sufficient to, degrade at least a portion of the contaminating RNA in the biological sample; c. combining the at least one RNase from the reagent article with the biological sample so as to form a solution thereof; d. incubating the solution with the at least one RNase for a period of time and under conditions sufficient to degrade at least a portion of the contaminating RNA; and e. separating the degraded RNA in the solution from the DNA.

The reagent article of this invention can be used with a wide variety standard biological kits, such as molecular biological kits for isolating DNA, where a known quantity of RNase(s) is needed having a relatively consistent and stable activity. Because the reagent article comprises lyophilized RNase(s), the reagent article will have a relatively long and stable shelf life. Because the reagent article comprises a premeasured single use quantity of the lyophilized RNase(s), the reagent article is relatively easy to use and can minimize potential errors due to user variability. The reagent article of this invention is useful in a wide variety of biological methods, including, for example, those for isolating DNA, removing RNA contaminants from DNA, and analyzing RNA in a biological sample. The reagent article can be used with existing commercially available kits, or can incorporated into existing or new kits with other components, for use in a wide variety of biological methods. The methods of this invention can provide straightforward, relatively easy to use and reproducible vehicles for isolating DNAs from biological samples contaminated with RNA.

### DETAILED DESCRIPTION

### 1. DEFINITIONS

As used herein, the term “biological sample” refers to any specimen or sample containing substances of biological or biochemical origin, including but not limited to whole tissues or portions thereof, cultured cells, whole blood or blood cells, other body fluids such as urine, sputum, semen and other secretions, fungal and plant tissues, bacterial cells, samples containing microorganisms and viruses, as well as biological or biochemical substance dissolved or suspended in aqueous solution or a liquid organic solvent.

As used herein, the term “RNA” is used generally to refer to any molecule comprising ribose nucleotides, including fully polymerized RNAs, as well as any oligionucleotide fragment thereof, and encompasses the various types of RNA known as mRNA, rRNA, tRNA and hnRNA.

As used herein, the term “DNA” is used generally to refer to any molecule comprising deoxyribose nucleotides, including fully polymerized DNAs, as well as any oligionucleotide fragment thereof.

As used herein, the terms “ribonuclease” and “RNase” are used interchangeably to refer to those nucleases that degrade RNA into its respective constituent nucleotides or fragments. Examples of such RNases include RNase A, RNase H, RNase I, RNase T1, RNase III, as well as mixtures of these RNases.

As used herein, the term “lyophilized” refers to any composition that has been freeze-dried, typically from an aqueous solution.

As used herein, the term “premeasured single use quantity” refers to an amount of the lyophilized RNase, as well as any other component or compositions included in the reagent, that has been measured to provide a given desired quantity suitable typically for a single use thereof. The premeasured quantity of lyophilized RNase can be in the form of a loose form such as a powder or can be in compacted form such as a pellet, tablet or capsule.

As used herein, the term “holder” refers to any device that is capable of storing, retaining, supporting or providing a premeasured quantity of the lyophilized RNase, as well as any other component included in or with the reagent article, until used. Suitable holders for use herein can be in the form of containers, vessels, receptacles, packages, or supporting substrates. The holder can also be used for carrying out the various uses involving the lyophilized RNase(s) (e.g., as a reaction vessel). The holder can be sealed or provided with a removable closure such as a cap or lid. Alternatively, the holder can be provided with a reagent compartment or chamber containing the lyophilized RNase that is separated from the “reaction” compartment or chamber of the holder by frangible or friable divider, partition or seal so that the lyophilized RNase can be released into the “reaction” chamber/compartment containing the biological sample of interest when the holder is centrifuged or otherwise shaken to break the divider or seal so as to combine and mix the RNase with the sample. See U.S. Pat. No. 5,643,767 (Fishchetti et al), issued Jul. 1, 1997, which is incorporated by reference. Alternatively, the divider, partition or seal can be one that can be broken or ruptured such as by chemicals, enzymes or heat, for example, a wax that can be digested upon exposure to enzymes or a cellulose membrane that is degraded by weak acids. Suitable holders can also simply provide a support (e.g., a substrate) for the premeasured quantity of lyophilized RNase until use. Some specific examples of holders suitable for use herein include, but are not limited to vials, tubes, supporting sticks, strips or swabs made of plastic or other suitable solid material (i.e., a “dipstick”) that can also serve as a mixer or stirrer, “blister”-type packages, and the like. The holder can be in the form of a single unit, for example, a single vial, tube, etc., or can be associated together as a plurality of units, such as a plurality of tubes connected to and detachable from a temporary support structure, or a plurality of dipsticks, pellets, tablets or capsules in a “blister” type package to provide separated premeasured quantities of lyophilized RNase(s), as well as any other component included in or with the reagent article. Particularly suitable holders for use herein are those capable of being used to carry out the desired use of the reagent article such as a capped centrifuge tube (e.g., an Eppendorf microcentrifuge tube).

As used herein, the term “tube” refers to an elongated, typically cylindrical structure along at least a portion thereof that is closed or sealed at least one end thereof, e.g., a test tube, centrifuge tube, and the like.

As used herein, the term “buffer” refers to any compound, composition or aqueous solution thereof that changes pH only slightly or minimally when acids or bases are added thereto. Representative buffers for use herein include, but are not limited to sodium phosphate mono basic, sodium phosphate dibasic, potassium hydrogen tartrate, potassium dihydrogen citrate, potassium hydrogen phthalate, sodium tetraborate, sodium carbonate, sodium bicarbonate, tris(hydroxymethyl)aminomethane (Tris), 2-N-morphilinoethanesulfonic acid (MES), N-2-acetamido-2-iminodiacetic acid (ADA), as well as various combinations of buffering agents including but not limited to sodium dihydrogen phosphate and disodium monohydrogen phosphate; disodium monohydrogen phosphate and sodium chloride; sodium carbonate, sodium bicarbonate and sodium chloride; potassium dihydrogen phosphate and sodium monohydrogen phosphate potassium hydrogen tartrate and potassium dihydrogen phosphate; acetic acid and sodium acetate; citric acid and sodium hydroxide; potassium hydrogen phthalate and sodium hydroxide; potassium hydrogen phosphate and sodium phosphate; Tris and hydrochloric acid; sodium tetraborate and hydrochloric acid; glycine and hydrochloric acid; triethanolamine and hydrochloric acid; Tris and sodium hydroxide; and the like. The buffer or solution thereof is usually in an amount sufficient to provide a pH in the range of from about 7 to about 12, typically in the range of from about 7 to about 10 and more typically in the range of from about 7 to about 8.

As used herein, the term “liquid organic solvent” usually refers to liquid alcohols (monohydric or polyhydric) and liquid polar aprotic solvents. Alcohols suitable for use herein include, but are not limited to, lower alcohols of from 1 to 10 carbon atoms such as methanol, ethanol, isopropanol, n-propanol, t-butanol, as well as glycerol, propylene glycol, ethylene glycol, polypropylene glycol and polyethylene glycol, and more typically isopropanol and ethanol. Polar aprotic solvents suitable for use herein include, but are not limted to, dimethyl sulfoxide (DMSO), dimethyl formamide (DMF), N-methypyrrolidone (NMP), tetrahydrofuran (THF), dioxane, acetonitrile and the like.

As used herein, the terms “surfactant” usually refers to anionic, cationic, amphoteric, zwitterionic, and nonionic surfactants, as well as mixtures thereof. Representative surfactants suitable for use herein include, but are not limited to, sorbitan trioleate, sorbitan tristearate, propylene glycol monostearate, sorbitan sesquiolate, glycerol monostearate, sorbitan monooleate, propylene glycol monolaurate, sorbitan monostearate, diethylene glycol monostearate, diethylmonolaurate, sorbitan monopalmitate, sorbitain monolaurate, polyoxyethylene ethers, polyoxyethylene lauryl ether, polyoxyethylen sorbitan monolaurate, polyoxyethylene sorbitan monopalmitate, polyoxyethylene sorbitan monostearate, polyoxyethylene sorbitan monooleate, polyoxyethylene sorbitan tristearate, polyoxyethylene sorbitan trioleate, polyoxyethylene glycol monooleate, polyoxyethylene glycol monostearate, triethanolamine oleate, polyoxyethylene nonyl phenol, polyethylene glycol monolaurate, polyoxyethylene glycol monostearate, polyoxyethylene glycol monooleate, polyoxyethylene stearyl ether, polyoxyethylene oleyl ether, polyoxyethylene cetyl ether, polyoxyethylene stearate, sodium oleate, potassium oleate, cetyl ethyl morpholinium ethosulfate, sodium lauryl (dodecyl) sulfate, sodium caprylate, sodium caprate, sodium laurate, sodium myristate, sodium cholate, sodium desoxycholate, sodium dihydrocholate, tetradecyltrimethyl ammonium bromide, hexadecylpyridinium chloride, and mixtures thereof, as well as commercial compositions of these surfactants sold under the trade names SPANs, TWEENs (e.g., TWEEN 20, 30 and 80) and TRITONs (e.g., TRITON X-100 and X-114). Suitable levels of surfactant for use herein are typically in the range of from about 0.001 to about 7.5%.

As used herein, the term “salts” refers to a compound produced by the interaction of an acid and a base. Salts suitable for use herein include, but are not limited to, those prepared from the following acids: hydrochloric, hydrobromic, sulfuric, nitric, phosphoric, maleic, acetic, salicylic, p-toluene sulfonic acid, tartaric, citric, methane sulfonic, formic, malonic, succinic, naphthalen-2-sulfonic, and benzene sulfonic. Exemplary salts include, but are not limited to, sodium chloride, potassium phosphate, sodium acetate, and sodium bicarbonate. Salt concentrations suitable for use herein are typically in the range of from about 0.01 to about 1 M, more typically in the range of from about 0.1 to about 0.5 M.

As used herein, the term “chaotropic agent” refers to a substance that causes disorder in a protein or nucleic acid by, for example, but not limited to, altering the secondary, tertiary, or quaternary structure of a protein or a nucleic acid while leaving the primary structure intact. Representative chaotropic agents suitable for use herein include, but are not limited to, chaotropically effective amounts of guanidine thiocyanate, guanidine hydrochloride, sodium iodide, sodium perchlorate, urea, hydroxides such as sodium or potassium hydroxide, guanidine salt, potassium thiocyanate, formamide, sodium chloride, and mixtures thereof. Particularly suitable chaotropic agents include solutions of guanidine thiocyanate, guanidine hydrochloride, sodium iodide and mixtures thereof in concentrations in the range of from about 1 to about 12 M, typically from about 1 to about 7 M and more typically from about 2 to about 5 M.

As used herein, the term “lysis agent” refers to an agent capable of partially or completely disrupting cell membranes, thus breaking open cells or groups of cells so that the intracellular components thereof, including DNA, are released into the surrounding medium. Suitable lysis agents for use herein include alkaline lysis agents involving an alkali or base such as sodium hydroxide that tend to incompletely disrupt cell membranes, as well as enzymatic lysis agents that tend to more completely disrupt cell membranes.

As used herein, the term “comprising” means various compositions, components, compounds, enzymes and steps can be conjointly employed in the present invention. Accordingly, the term “comprising” encompasses the more restrictive terms “consisting essentially of” and “consisting of.”

All amounts, parts, ratios and percentages used herein are by weight unless otherwise specified.

The reagent article of this invention comprises the holder and a premeasured single use quantity of at least one lyophilized RNase associated with the holder. The particular premeasured single use quantity of lyophilized RNase(s) included will depend on a variety of factors including the RNase(s), the other components or compositions included and the particular intended use or uses to be made of the reagent article. Typically, the reagent article comprises a premeasured single use quantity of lyophilized RNase(s) in the range of from about 0.5 to about 20 mg, more typically from about 1 to about 2 mg. In addition to the lyophilized RNase(s), the reagent article can also optionally include a premeasured single use quantity of other reagents, such as buffers, salts and mixtures thereof.

The reagent article of this invention can be packaged and distributed as a single product or as a plurality of such products having either the same premeasured single use quantity of lyophilized RNase(s), or different premeasured single use quantities of lyophilized RNase(s). The reagent article of this invention can be provided separately as a stand alone product, or can be provided as part of a kit that includes other components such as other reagents including, but not limited to, buffers, chaotropic agents, lysis agents (e.g., alkaline lysis agents and/or enzymatic lysis agents), and liquid organic solvents, as well as reagent equipment including, but not limited to, vials, tubes, filters, bags, etc. The other reagents such as the buffers, chaotropic agents and lysis agents can be provided in bulk quantities as solids or liquids to be used or measured out as needed, or can be provided in premeasured single use quantities for convenience and ease of use. An embodiment of the kit of this invention can comprise: (a) at least one reagent article as a component thereof; (b) at least one other reagent component selected from the group consisting of buffers, salts, chaotropic agents, lysis agents and liquid organic solvents; and (c) at least one equipment component selected from the group consisting of tubes and filters. A particularly suitable kit for use in removing contaminating RNA from a biological sample containing DNA comprises: (a) a plurality of reagent articles wherein the holder is a centrifuge tube containing the RNase(s); (b) buffers, salts, chaotropic agents and lysis agents as the other reagent component, typically as prepared solutions thereof; and (c) at least a plurality of filters as the equipment component. The plurality of reagent articles can comprise one premeasured single use quantity of the RNase(s) or can comprise two or more different premeasured single use quantities of the RNase(s).

The reagent article, or kit containing same, can also be provided with a set of instructions for how to use the reagent article, and other components in the kit. These instructions can be written or printed on the packaging that the reagent article or kit is sold or distributed in or with, or on a sheet(s) of paper separately therefrom. Alternatively, the reagent article or kit can include computer software (e.g., in the form of a floppy disk(s), CD ROM disk(s) or other non-volatile electronic storage media) packaged or otherwise associated with the article or kit that provides the instructions on how to use the reagent article, as well as the other components in the kit, or the instructions can be provided and obtained electronically (e.g., via the Internet) from a remote site, such as from a web site or computer server.

Besides the convenience of premeasured aliquots of RNase(s), the reagent article of this invention decreases sample variability due to user error. This assumes that the process for preparing the reagent article is reproducible in providing relatively accurate quantities of the lyophilized RNase(s), as well as any other components included with the article. In addition, the lyophilized RNase has extended shelf life in a useful storage form, thereby maximizing the activity of the enzyme and increasing its value as a component in a convenient kit format.

The reagent article, with or without an associated kit, is useful in a wide variety of applications requiring a premeasured single use quantity of RNase. These applications include, but are not limited to, methods for isolation of DNA, and for removing RNA as a contaminant from extrachromosomal DNA isolates such as plasmids, cosmids, or BACS. See, for example, the methods for isolating extrachromosomal nucleic acids from biological materials disclosed in International Patent Application WO 00/77235 (Reed et al), published Dec. 21, 2000, which is incorporated by reference. Other alternative uses include methods involving digestion of RNA for the purpose of analyzing gene expression or RNA mapping, structure and/or function, such as an S1-nuclease Protection Assay (S1), or Ribonuclease Protection Assay (RPA).

An embodiment of a method for removing contaminating RNA from a biological sample containing DNA can comprise the steps of: a. providing a biological sample containing DNA and contaminating RNA; b. providing a single use reagent article wherein the lyophilized RNase(s) is capable of, and in a premeasured single use quantity sufficient to, degrade at least a portion of the contaminating RNA in the biological sample; c. combining the at least one RNase from the reagent article with the biological sample so as to form a solution thereof; d. incubating the solution with the at least one RNase for a period of time and under conditions sufficient to degrade at least a portion of the contaminating RNA; and e. separating the degraded RNA from the DNA in the solution.

Prior to step (a), the biological sample is typically subjected to treatment with a lysis reagent to prepare the sample. A liquid organic solvent such as isopropanol is also typically added during step (e). The DNA obtained after step (e) is usually washed with a liquid organic solvent, typically comprising ethanol. If desired, steps (c) through (e) can be repeated one or more times to remove additional contaminating RNA.

Another embodiment of the method of this invention that is particularly suitable for isolating DNA in a biological sample also containing contaminating RNA using centrifugation techniques comprises at least the following initial steps of: a. providing a buffered solution of the biological sample containing DNA and contaminating RNA; b. adding the buffered solution (e.g., Tris) to a reagent article comprising: a centrifuge tube; and at least one lyophilized RNase in the container that is capable of, and in a premeasured single use quantity sufficient to, degrade at least a portion of the contaminating RNA in the buffered solution; c. after step (b), incubating the buffered solution for a period of time and under conditions sufficient to degrade at least a portion of the contaminating RNA; d. after step (c), adding an effective amount of a chaotropic agent (e.g., isopropanol, sodium iodide and guanidine thiocyanate) to the buffered solution to release at least a portion of the DNA; e. after step (d), adding a portion of liquid organic solvent (e.g., isopropanol) to the buffered solution; f. after step (e), centrifuging the buffered solution to form a supernatant liquid comprising the degraded RNA, and a residual solid comprising the released DNA; and g. separating the supernatant liquid from the residual solid.

Prior to step (a), the biological sample is typically subjected to treatment with a lysis reagent to prepare the sample. To further purify and isolate the DNA, it can be desirable to wash (step h) the residual solid of step (g) with a second portion of liquid organic solvent (typically ethanol), as well as carry out the following additional steps of: i. providing a second reagent article comprising: (1) a second centrifuge tube; and (2) at least one lyophilized RNase in the second tube that is capable of, and in a second premeasured single use quantity (can be the same or different from the first premeasured quantity) that is sufficient to, degrade at least a portion of the remaining contaminating RNA in the washed solid; j. adding a second buffered solution to the second tube to suspend the second quantity of at least one RNase in the second buffered solution; k. adding the second buffered solution to first tube containing the residual solid; l. after step (k), incubating the second buffered solution for a period of time and under conditions sufficient to degrade at least an additional portion of the remaining contaminating RNA; m. after step (l), adding a second effective amount of a chaotropic agent to the second buffered solution to release additional DNA; n. after step (m), adding a third portion of liquid organic solvent to the second buffered solution; and o. after step (n), centrifuging the second buffered solution to form a second supernatant liquid comprising the additional degraded RNA, and a second residual solid comprising the combined released DNA; and p. separating the second supernatant liquid from the second residual solid, with or without additional washing of the second residual solid with a liquid organic solvent.

In preparing the reagent article of this invention, the RNase or mixture thereof can be solubilized in an aqueous solution, such as water, or water containing physiological concentrations of buffer and salt. An aliquot of the RNase solution can be placed in a tube or other container of varying size or composition according to desired application or use. For example, for many molecular biological applications, a 1.5 or 2.0 ml polypropylene centrifuge tube with a screw cap or an attached snap-cap closure is suitable as the holder for the lyophilized RNA. A glass tube, or tube composed of other materials can also be used, when appropriate. A larger volume tube can also be used for some applications requiring a larger quantity of RNase, such as larger-scale protocols for isolating DNA, or for analysis of RNA.

The RNase solution in the tube is lyophilized such that the liquid evaporates completely or substantially completely from the tube. The tube containing the resulting lyophilized RNase can be stored at room temperature, or refrigerated or frozen for future use to enhance shelf life and storage flexibility when packaged in a kit. It is expected or intended that the tube containing the lyophilized RNase(s) will be used one time and then discarded. In, for example, DNA isolation or purification methods, the RNase lyophilized in the tube should be of a suitable quantity to degrade a range of RNA contamination that is typically carried over in DNA isolation techniques widely used in the art. For maximum convenience, the user can simply add to the tube of lyophilized RNase(s) a solution that already contains the DNA isolate requiring removal of RNA. Alternatively, a DNA isolate requiring purification can be added after the RNase(s) has been resuspended in the lyophilized tube, or is resuspended in another tube or container. In either case, another advantage of this invention is that suitable components from a wide variety of commercial kits can be used to resuspend the lyophilized RNase, thus allowing the lyophilized RNase tube to be used with established protocols or existing kits.

The following four alternative options for resuspending the lyophilized RNase tubes can be employed in the practice of this invention.

1. A solution of RNase in water is placed in a tube and lyophilized. The lyophilized RNase in the single-use tube is resuspended by adding water. This provides the user with a premeasured unit of RNase in water.

2. A solution of RNase in water is placed in a tube and lyophilized. The lyophilized RNase in the single-use tube is resuspended by adding an aqueous buffered salt solution from a commercial kit, e.g., one typically used for isolating DNA. This provides the user with a premeasured unit of RNase in a buffered salt solution.

3. A buffered salt solution of the RNase is lyophilized in the single use tube, and is then resuspended by adding water to the tube. This provides the user with a premeasured unit of RNase in a buffered salt solution, but with the convenience of just adding water.

4. The RNase is lyophilized in the single use tube from water or a buffered salt solution. A solution containing the DNA to be treated is directly added to the lyophilized RNase in the tube, thus resuspending the RNase in the presence of the DNA.

In practicing any one of the first three options described above (and after the lyophilized RNase has been resuspended), the resuspended RNase solution can be transferred to another tube containing a precipitated DNA pellet or a DNA solution that the user wishes to treat. Alternatively, a solution containing the DNA can simply be added to the resuspended RNase in the single use tube. In addition to usefulness as part of a DNA isolation method, the fourth option provides a convenient way to further purify any stocks of DNA the user may already have on hand that are found to contain RNA contaminants.

With any of these methods for removing RNA contaminants from DNA, the solution of RNase and DNA is typically incubated for from about 10 to about 60 minutes, usually at a temperature of from about 20° to about 65° C. (typically in the range of from about 37° to about 57° C.) to promote degradation of the RNA contaminants.

### EXAMPLES

The following examples illustrate various embodiments of the reagent article of this invention and methods for its use.

### Example 1

### Removing RNA Contaminants from DNA Sample

1. Place up to 500 μl of a DNA solution into a reagent article in the form of a single-use centrifuge tube containing a lyophilized RNase, e.g., RNase A or a mixture of RNase A and RNase T1. Gently mix by inverting approximately 20 times. Incubate for 30 minutes in a 37° C. water bath. Every 10 minutes during the incubation, mix gently by inverting 20 times.

2. Add 800 μl of a chaotropic solution containing 17% isopropanol, 3M sodium iodide and 1M guanidine thiocyanate to the tube to dissolve the RNase.

3. Add 600 μl of isopropanol to the tube from step 1. Mix by gently inverting approximately 20 times. Centrifuge at maximum speed for 5 minutes, and then pour off and discard the supernatant.

4. Wash the residual DNA pellet from step 2 once using 70% ethanol: Add 1 ml of 70% ethanol and mix by gentle inversion. Centrifuge at maximum speed for 2 minutes and the carefully pour off and discard supernatant. Pipette off any residual ethanol and allow the residual DNA pellet to air dry for 5 minutes.

5. Resuspend dried pellet from step 3 in the desired volume of solvent (e.g., water, buffered salt solution, or other aqueous solution) appropriate for the application of interest.

### Example 2

### DNA Isolation Method

This example illustrates use of a single-use lyophilized RNase tube of this invention in an existing method for isolating DNA. The protocol for this method is from a commercially available kit (Spin Doctor; Gerard Biotech; Cincinnati, Ohio). Centrifuge tubes containing 100 μl of lyophilized RNase are prepared from a 70% or 17.5% mixture of RNase A (130 Kunitz units) and RNase T1 (100 Kunitz units) in nuclease free sterile water. The tubes containing the RNase mixture are placed in a lyophilizer and dried at less than −40° C. for at least 10 hours under a vacuum of 100 μ (+25 μ) to completion. The tubes containing the lyophilized RNase are capped and provided in this form to the end user until resuspension in steps 11 and 16 that are described hereafter.

Steps 1 through 10 involving treatment with an alkaline lysis agent for initial sample preparation (for 100-125 ml sample) are as follows:

1. Separate the bacterial growth from the media by centrifuging at 4,000 g for 10 minutes at 4° C. Discard the supernatant.

2. Re-suspend residual cell pellet in 10 ml of an aqueous 50 mM Tris buffer solution, pH 8.0. Using a serological pipette, gently stir/disrupt the cell pellet into manageable “chunks.” Gently draw the buffered aqueous Tris solution/cell mixture in and out of the pipette until the cell pellet is completely mixed. Ensure that there are no clumps remaining. For gram-positive bacteria, add lysozyme to the re-suspended mixture in a concentration of 4 mg/ml. Mix by gently inverting 20 times and allow mixture to incubate at room temperature for 30 minutes.

3. Add 10 ml of an aqueous solution of 200 mM sodium hydroxide and 1% sodium dodecyl sulfate, and mix by gently inverting approximately 20 times.

4. Add 10 ml of an aqueous solution of 11.5% acetic acid and 3M potassium acetate, and mix by gently inverting approximately 30 times.

5. Centrifuge at 8,000-13,000 g for 10 minutes at 4° C.

6. After step 5 is completed, pour the supernatant through a cell strainer into a clean centrifuge tube.

7. Add 30 ml of isopropanol to the supernatant from step 6, and mix by gently inverting approximately 20 times.

8. Centrifuge at 8,000-13,000 g for 10 minutes at 4° C.

9. Pour off and discard the supernatant. Turn the tube so that the pellet is out of the remaining liquid and pipette off the excess supernatant. Allow to air dry for 15 minutes. If isopropanol remains in the tube after the air-drying, pipette once more to remove it.

10. Re-suspend residual DNA/RNA pellet from step 9 in 500 μl of an aqueous 10 mM Tris buffer solution, pH 8.0.

11. Add buffered solution of resuspended DNA/RNA pellet to a first single use tube containing the 70% mixture of lyophilized RNases.

12. Cap the first tube and gently mix by inverting approximately 20 times. Incubate for 30 minutes in a 37° C. water bath. Every 10 minutes during the incubation, mix by gently inverting 20 times.

13. Add 800 μl of a chaotropic solution containing 17% isopropanol, 3M sodium iodide and 1M guanidine thiocyanate to the first tube. Mix by gently inverting approximately 20 times.

14. Add 600 μl of isopropanol to the first tube. Mix by gently inverting approximately 20 times. Centrifuge at maximum speed for 5 minutes, and pour off and discard the supernatant.

15. Wash the residual DNA pellet from step 14 once using 70% ethanol: Add 1 ml of 70% ethanol and gently invert 20 times. Centrifuge at maximum speed for 2 minutes, and carefully pour off and discard the supernatant. Pipette off any residual ethanol and allow the pellet to air dry for 5 minutes.

16. Add 500 μl of the buffered aqueous Tris solution of step 10 to a second single use tube containing the 17.5% mixture of lyophilized RNases, mix by inverting 10 times, and then vortex for 3 seconds to resuspend the RNase in the solution. Transfer the RNase solution from the second tube into the first tube containing the dried pellet from step 15. Partially re-suspend the pellet in the RNase solution by gently pipetting the liquid up and down approximately 20 times, using a wide bore 1000 μl tip to avoid shearing. Incubate for 15 minutes in a 37° C. water bath, and mix gently by inversion every 5 minutes.

17. Add 800 μl of the chaotropic solution of step 13 to the second tube, and mix gently by inversion.

18. Add 600 μl of isopropanol to the second tube, and mix gently by inversion. Centrifuge at maximum speed for 5 minutes, and pour and discard the supernatant.

19. Partially re-suspend the pellet in the second tube by adding 900 μl of the chaotropic solution of step 13.

20. Gently pipette solution in the second tube up and down with a 1000 μl wide bore pipette tip 8-10 times to re-suspend the pellet, followed by inversion.

21. Add 450 μl of isopropanol to the second tube, and mix gently by inversion. Centrifuge at maximum speed for 3 minutes. Pour off and discard the supernatant.

22. Partially resuspend the residual pellet in the second tube from step 21 in 900 μl of a chaotropic solution containing 17% isopropanol and 2M sodium iodide. Pipette the solution up and down gently 8-10 times with a 1000 μl wide bore pipette, and then mix gently by inversion. Add 450 μl of isopropanol to the second tube. Centrifuge at maximum speed for 3 minutes. Pour and discard the supernatant, and then pipette off any residual.

23. Partially re-suspend the residual pellet in the second tube from step 22 in 500 μl of the aqueous Tris buffer solution of step 10 by gently pipetting the solution up and down 8-10 times with a 1000 μl wide bore tip pipette.

24. Add 50 μl of an aqueous 3M sodium acetate solution to the second tube, and then mix gently by inversion.

25. Add 500 μl of isopropanol to the second tube, and then mix gently by inverting 20 times. Centrifuge for 3 minutes at maximum speed. Pour off and discard the supernatant, and then pipette off any residual liquid.

26. Wash the residual DNA pellet in the second tube from step 25 twice using 70% ethanol. Add 1 ml of 70% ethanol and gently invert several times to mix. Centrifuge at maximum speed for 2 minutes, and pour off and discard the supernatant.

27. Pipette off any residual ethanol and allow to air dry for 10 minutes.

28. Resuspend the DNA pellet in 10 mM Tris buffer solution, pH 8.0.

### Example 3

### Variation of Example 2

Steps 1 to 14 are the same as Example 2. Steps 15 through 24 are replaced with the following steps 15 through 19:

15. Add 800 μl of an aqueous buffer solution of 15 mM sodium hydroxide to the first tube and resuspend pellet by gently pipetting 10-15 times using a wide bore 1000 μl tip and allow to incubate for 5 minutes at 57° C. Pipette 10-15 times after incubation to further re-suspend pellet.

16. Add 100 μl of the buffered aqueous 500 mM Tris solution to a second single use tube containing the 70% mixture of lyophilized RNases, mix by inverting 10 times, vortex briefly, invert and vortex briefly, and centrifuge at full speed for 10-15 seconds to resuspend the RNase in the solution. Add RNase solution to first tube with re-suspended pellet. Incubate for 10 minutes in a 37° C. water bath, and mix gently every 5 minutes by inverting 20 times.

17. Add 90 μl of 3M sodium acetate solution to first tube and mix gently by inverting 20 time.

18. Add 900 μl of isopropanol to the first tube and mix gently by inverting 20 times. Centrifuge at maximum speed for 5 minutes and pour off and discard supernatant

19. Add 500 μl of buffered aqueous 500 mM Tris solution to teh first tube. Partially re-suspend the pellet by pipetting up and down gently 8-10 times with a 1000 μl wide bore pipette, and allow to incubate for 2 minutes at 57° C.

Add 100 μl of a chaotropic solution containing 17% isopropanol and 3M sodium iodide to the first tube, and mix by inverting 30 times. Add 600 μl of isopropanol to the first tube and centrifuge at maximum speed for 5 minutes. Pour supernatant off slowly and discard.

20. Carry out steps 25 through 28 of Example 2.

### Example 4

### Isolating BAC DNA

This protocol is useful for isolating BAC DNA. It is taken from a commercially available kit, also from Gerard Biosciences, and requires an additional 15 minute RNase digestion after re-suspending in the aqueous Tris buffer solution:

1. Re-suspend cell pellet in 10 ml of an aqueous 50 mM Tris buffer solution, pH 8.0, as in step 2 of Example 2.

2. Add 1 ml of an aqueous 10 mM Tris buffer solution, pH 8.0, to a first single use tube containing the 17% mixture of lyophilized RNases, mix by inverting approximately 10 times, then Vortex for 3 seconds to resuspend RNase. Add the resuspended RNase solution directly into buffered Tris solution/pellet mixture from step 1, and then mix by gently swishing 20 times. Incubate at room temperature for 15 minutes, swishing every 5 minutes.

3. Carry out steps 3 through 28 of Example 2.

### Example 5

### Analyzing for mRNA in Cell Sample

This example illustrates a method for using single use lyophilized RNase tubes of this invention to analyze for the abundance of an mRNA of interest in a population of cells. The lyophilized RNase tubes contain 0.28 μl of a mixture of RNase A (130 Kunitz units) and RNase T1 (100 Kunitz units) in a 1:1 weight ratio that is lyophilized with 200 μl of an RNase digestion buffer.

1. Dry down 10 to 50 μg of RNA for each sample.

2. Determine total number of samples and prepare hybridization buffer cocktail for n+1 samples (1× Hybridization buffer comprises 80% formamide, 40 mM PIPES, pH6.6, 1 mM EDTA, and 0.4M NaCl) as follows: Hybridization Cocktail (per Reaction): 2 μl 10× Hybridization buffer 2 μl mixture of riboprobes 16 μl deionized formamide The riboprobes are prepared by adding approximately 15,000 dpm of each freshly synthesized riboprobe for the mRNA of interest, and 25,000 dpm for an L32 or other control mRNA to each reaction tube. The concentrated riboprobes are counted and diluted and combined in a final volume of 2 μl per reaction.

3. Resuspend dry RNA samples in 20 μl hybridization cocktail by vortexing.

4. Heat tubes containing samples to at least 80° C. for 5 minutes to denature the RNA and probes. Then transfer directly to optimal temperature water bath for hybridization overnight or for 16 to 18 hours. Optimal temperature is determined empirically for each probe by testing several temperatures in the range of 50° to 65° C.

5. Prepare a sufficient number of lyophilized RNase tubes of this invention on ice for all samples by adding 200 μl of water to each tube. Transfer 200 μl ice cold RNase solution to each mRNA hybridization tube; each is individually removed from the water bath, and mixed before placing on ice. Keep mixed tubes on ice until all samples are processed.

6. Vortex samples to mix, centrifuge briefly to collect the reaction in the bottom of the tube and incubate at 33° C. for 1 hour.

7. Stop each reaction by adding 225 μl RNase inactivation/precipitation solution (Ambion, catalog#8539G1), 100 μl 100% ethanol, and 0.25 μl glycoblue (Ambion #9516). These reagents can be mixed to form a cocktail and added at 325 μl per sample. Mix well and incubate the rack at −20° C. for at least 30 minutes to allow protected fragments to precipitate.

8. Centrifuge the precipitates at full speed for 15 min. Remove the supernatant and discard as radioactive waste.

9. Wash the pellets with approximately 300-500 μl 70% ethanol. This step can be repeated, if necessary, to remove excess salt from the pellet. Dry briefly under vacuum.

10. Resuspend in 4 μl gel loading buffer by vortexing and spin down briefly. Heat to denature at 90° C. for 3 to 5 minutes and load all of each sample in a 6% acrylamide/8M urea gel. Run using 75 watts as the limiting factor on the power supply until the bromphenol blue of the loading dye is ½ to ¾ the way down a 40 cm gel (approx. 55 minutes). Separate the plates and trim the gel. Fix the gel in 10% methanol/10% acetic acid for two—5 minute washes. Drain, and lift gel with 3M paper cut slightly bigger than the gel. Cover the gel with saran wrap, cut a second piece of 3M paper and dry the gel in the gel dryer at 80° C.

11. Remove saran wrap and expose to film at −80° C. with screen overnight. Quantify bands by phosphorimaging using the Storm system and Imagequant software.

### Example 6

### Alternative Forms

In place of the tubes containing the premeasured single use quantity of lyophilized RNase(s) in Examples 1 through 4, single use quantities of the RNase(s) can be provided in alternate forms, such as lyophilized RNase(s) dried on a solid (e.g., plastic) “dipstick” as the substrate (with or without additional components such as buffers), or can be provided in the form of separate pellets or capsules, or as a plurality of pellets or capsules as part of, for example, a “blister pack” from which individual pellets or capsules can be dispensed as needed. Any of these alternate forms can also incorporate other components, such as buffers and salts, that are used in preparing the solutions of biological samples and/or RNase(s). The “dipstick” embodiment of this invention can be placed in a tube containing the solution of DNA and contaminating RNA, and allowed to soak until the RNase(s) and any buffer components are dissolved from the surface of the plastic substrate. The pellet or capsule of lyophilized RNase(s) can be added to a tube containing the solution of DNA and contaminating RNA, with the tube being capped, and inverted to resuspend the RNase(s) and any buffer components. Incubation and DNA isolation could then proceed as described in Examples 1-4.

1. A reagent article, which comprises:
a holder; and
a premeasured single use quantity of at least one lyophilized RNase associated with the holder.

2. The article of claim 1 wherein the holder is selected from the group consisting of vials, tubes, sticks, strips and swabs.

3. The article of claim 2 wherein the holder is a centrifuge tube.

4. The article of claim 1 wherein the at least one RNase is selected from the group consisting of RNase A, RNase H, RNase I, RNase T1, RNase III, and mixtures thereof.

5. The article of claim 4 wherein the at least one RNase comprises a mixture of RNase A and RNase T1.

6. The article of claim 1 which further comprises another reagent selected from the group consisting of buffers, salts and mixtures thereof.

7. A biological kit, which comprises: a. at least one reagent article comprising: a holder; and a premeasured single use quantity of at least one lyophilized RNase associated with the holder; and b. at least one other component selected from the group consisting of other reagent components and reagent equipment.

8. The kit of claim 7 wherein the holder is a centrifuge tube containing the at least one RNase.

9. The kit of claim 8 wherein the at least one other reagent component is selected from the group consisting of buffers, salts, chaotropic agents, lysis agents and liquid organic solvents; and wherein the at least one equipment component is selected from the group consisting of tubes and filters.

10. The kit of claim 9 which comprises a plurality of reagent articles, and wherein the at least one other reagent component comprises buffers, salts, chaotropic agents and lysis agents, and wherein the at least one equipment component comprises a plurality of filters.

11. The kit of claim 9 which further comprises instructions for use associated therewith.

12. The kit of claim 11 wherein the instructions describe a method for removing contaminating RNA from a biological sample containing DNA.

13. The kit of claim 11 wherein the instructions are written on at least one sheet of paper.

14. The kit of claim 11 wherein the instructions are provided on computer software.

15. A method for removing contaminating RNA from a biological sample containing DNA, the method comprising the steps of:
a. providing a biological sample containing DNA and contaminating RNA;
b. providing a single use reagent article comprising: an RNase holder; and at least one lyophilized RNase associated with holder that is capable of, and in a premeasured single use quantity sufficient to, degrade at least a portion of the contaminating RNA in the biological sample;
c. combining the at least one RNase from the reagent with the biological sample so as to form a solution thereof;
d. incubating the solution for a period of time and under conditions sufficient to degrade at least a portion of the contaminating RNA; and
e. separating the degraded RNA in the solution from the DNA.

16. The method of claim 15 wherein the holder is a centrifuge tube that contains the at least one RNase, and
wherein steps (3) and (4) are carried out in the tube.

17. The method of claim 15 wherein steps (c) through (e) are repeated out one or more times.

18. The method of claim 15 wherein the biological sample is subjected to treatment with a lysis agent prior to step (a).

19. A method for isolating DNA in a biological sample containing contaminating RNA, which comprises the steps of:
a. providing a buffered solution of the biological sample containing DNA and contaminating RNA;
b. adding the buffered solution to a reagent article comprising: a centrifuge tube; and at least one lyophilized RNase in the tube that is capable of, and in a premeasured single use quantity sufficient to, degrade at least a portion of the contaminating RNA in the buffered solution;
c. after step (b), incubating the buffered solution for a period of time and under conditions sufficient to degrade at least a portion of the contaminating RNA;
d. after step (c), adding an effective amount of a chaotropic agent to the buffered solution to release at least a portion of the DNA;
e. after step (d), adding a portion of liquid organic solvent to the buffered solution;
f. after step (e), centrifuging the buffered solution to form a supernatant liquid comprising the degraded RNA, and a residual solid comprising the released DNA; and
g. separating the supernatant liquid from the residual solid.

20. The method of claim 19 wherein the biological sample is subjected to treatment with a lysis agent prior to step (a).

21. The method of claim 20 which comprises the further step (h) of washing the residual solid with a second portion of liquid organic solvent.

22. The method of claim 21 wherein the second portion of solvent comprises ethanol.

23. The method of claim 22 which comprises the further steps after step (h) of:
i. providing a second reagent article comprising: a second centrifuge tube; and at least one lyophilized RNase in the second tube that is capable of, and in a second premeasured single use quantity different sufficient to, degrade at least a portion of the remaining contaminating RNA in the washed solid;
j. adding a second buffered solution to the second tube to suspend the second quantity of at least one RNase in the second buffered solution;
k. adding the second buffered solution to first tube containing the residual solid;
l. after step (k), incubating the second buffered solution for a period of time and under conditions sufficient to degrade at least an additional portion of the remaining contaminating RNA;
m. after step (l), adding a second effective amount of a chaotropic agent to the second buffered solution to release additional DNA;
n. after step (m), adding a third portion of liquid organic solvent to the second buffered solution; and
o. after step (n), centrifuging the second buffered solution to form a second supernatant liquid comprising the additional degraded RNA, and a second residual solid comprising the combined released DNA; and
p. separating the second supernatant liquid from the second residual solid.

24. The method of claim 23 wherein the first and second quantity of at least one RNase each comprise a mixture of RNase A and RNase T1.

25. The method of claim 23 wherein the second quantity of at least one lyophilized RNase is a different from the first premeasured quantity of at least one lyophilized RNase.

26. The method of claim 24 wherein the first and second buffered solutions each comprise tris(hydroxymethyl) aminomethane.

27. The method of claim 26 wherein the first and third portions of solvent each comprise isopropanol.

28. The method of claim 27 wherein the first and second amounts of chaotropic agent each comprise a mixture of isopropanol, sodium iodide and guanidine thiocyanate.
